(19) United States
(12) Patent Application Publication
(10) Pub. No.: US 2010/0106065 A1
(43) Pub. Date: Apr. 29, 2010

(54) Orthotic Assembly for Selectively off-Loading a Weight-Bearing Joint

(76) Inventor: Michael J. Ward, Frisco, TX (US)

Correspondence Address: WILSON DANIEL SWAYZE, JR., 3804 CLEARWATER CT., PLANO, TX, 75025, US

(21) Appl. No.: 12/260,921

(22) Filed: Oct. 29, 2008

(51) Int. Cl. A61F 5/01

(52) U.S. Cl. 602/23

(57) ABSTRACT

An apparatus for supporting a calf of a user may include a first calf support member to support the calf of the user, a second calf support member to support the calf of the user, shoe member to support the first and second calf support member. The first calf support member and the second calf support member may cooperate to form a substantially continuous inner surface to support the calf of the user. The first calf support member may include a slot to accept the leg of the user, and the second calf support member may include a slot to accept the leg of the user. The first calf support member and the second calf support member may be detachably connected with a strap, and he first calf support member and the second calf support member may be detachably connected with a latch.

### FIELD OF THE INVENTION

The present invention relates to leg and foot brace devices that are secured to a patient's foot and leg.

### BACKGROUND OF THE INVENTION

Prior art devices of this type provide for a variety of leg, ankle and foot stabilization utilizing two-part brace assemblies interconnected by free hinges of an elastomeric materials as seen in U.S. Pat. No. 5,496,263 that permit the tolocural articulation to move about a single axis.

U.S. Pat. No. 5,486,157 discloses a hinge at the apex of a contoured heel portion to provide free dorsiflexion and plantar flexion with a pivot point below the hinges for inversion, eversion, pronation, and supination of the foot.

Another orthotic brace is illustrated in U.S. Pat. No. 5,086,760 which provides support to an anatomical joint that connects the lower leg with the foot that is selectively adjustable for predetermining the extent or angular range of dorsiflexion/plantar flexion and inverse/eversion of the foot. Such orthotic joints are also seen in U.S. Pat. Nos. 5,542,774 and 5,611,773.

In U.S. Pat. No. 5,542,774 a joint is illustrated having an interchangeable cam follower of shock absorbent material for more comfortable and therapeutic use.

In U.S. Pat. No. 5,611,773 multiple adjustable insert pins for an ankle pivot cap that allow for pin adjustment by movement within a contoured pivot path by corresponding registration with a dorsiflexion limiting aperture or plantar flexion limiting apertures.

A foot brace can be seen in U.S. Pat. No. 4,934,355 having a heel cup flexible connector to a leg engagement portion. The flexible connection is formed by an elastomeric block therebetween.

U.S. Pat. Nos. 5,088,479 and 5,593,383 disclose orthotic devices which are characterized by an adjustable foot portion interconnected to a leg portion by a semi-flexible heel member.

U.S. Pat. No. 5,593,383 introduces a foot pad with a securing apparatus to enhance and simplify usefulness.

U.S. Pat. No. 5,088,479 discloses a basic ankle and foot orthosis device having adjustable length and height for the leg and foot portion respectively.

In recent years, there has been a rapid growth in the popularity of sports and recreational activities amongst all age groups. Leisure and recreation has become an important aspect of a person's lifestyle. Concomitantly, there has been an increasing number of “weekend” athletes participating in gyms, tracks and courts, etc. some juveniles may have club feet or plantar fascitiis or other afflictions involving the ankle or foot.

Injuries to the bottom of this foot, ankle joint, lower leg, burns to the foot, tendon tears and knee injuries are commonly experienced by athletes and individuals who participate in a variety of different recreational sports, i.e., tennis, soccer, basketball or racquetball, as well as other activities. Damage to the ankle joint, lower leg, burns to the foot, tendon tears, plantar fascitiis and other afflictions can be quite painful causing the injured person to be unable to walk or run properly for a period of time. Because these injuries have become widespread, there is a need for an universal orthopedic device designed to provide to eliminate the weight from the ankle joint and foot so as to allow for immediate ambulatory mobility approaching 100% offloading to the injured joint and prevent further injury or allow the injured person to walk or even to continue his or her recreational activity.

In the past, athletes have relied on padding for injuries to the bottom of the foot in the event of injury thereto or to prevent injury. The padding does not eliminate the weight placed on the bottom of the foot, and consequently this type of padding does not allow the bottom of the foot to heal properly and does not prevent pain when walking or running.

Orthopedic devices presently do not provide an immediate solution to recovering patients or to the psychological benefit to an amputee that he might seem to be ambulatory.

### SUMMARY

An apparatus for supporting a calf of a user may include a first calf support member to support the calf of the user, a second calf support member to support the calf of the user, a shoe member to support the first and second calf support member.

The first calf support member and the second calf support member may cooperate to form a substantially continuous inner surface to support the calf of the user.

The first calf support member may include a slot to accept the leg of the user, and the second calf support member may include a slot to accept the leg of the user.

The first calf support member and the second calf support member may be detachably connected with a strap, and he first calf support member and the second calf support member may be detachably connected with a latch.

The strap may include a Velcro connection device, and the first calf support member may include a removable cushion member to cushion the calf of the user.

The second calf support member may include a removable cushion member to cushion the calf of the user, and the shoe member and the first calf support member may be connected with a support member.

The shoe member and the second calf support member may be connected with a support member, and the second calf support member may not extend to the shoe member.

The second calf support member may extend to the shoe member. And the apparatus may include a first thigh support member to support a thigh of the user.

The apparatus may include a second thigh support member to support the thigh of the user and to cooperate with the first thigh support member.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a perspective view of the an orthotic assembly providing mobility to a user during convalescence;

FIG. 2 illustrates a perspective view of the orthotic assembly of the present invention illustrating the offloading capability of the orthotic assembly;

FIG. 3 illustrates an exploded view of the orthotic assembly of the present invention;

FIG. 4 illustrates a sectional view of a lower portion of the second calf support member showing the lower portion of the second calf support member one;

FIG. 5 illustrates a cross-sectional view of the U shaped support member;

FIG. 6 illustrates a front perspective view of the orthotic assembly being used with an amputee;

FIG. 7 illustrates a rear perspective view of the orthotic assembly being shown in FIG. 6;

FIG. 8 is a diagrammatic side elevation of the first apparatus illustrating the complete isolation and securement of the ankle joint during ambulatory movement;

FIG. 9 illustrates an exploded perspective view of a second apparatus wherein the weight bearing means is a one piece component;

FIG. 10 illustrates a perspective view in partial cutaway of the foot bed component of FIG. 9 wherein the one-piece weight bearing means is an integrally formed with the foot bed structure;

FIG. 11 illustrates a perspective view of yet a third apparatus the orthotic assembly providing the mobility to the user with a permanent leg injury.

FIG. 12 illustrates an enlarge perspective in partial cutaway of FIG. 11 illustrating complete offloading of the ankle joint;

FIG. 13 illustrates an exploded view of the components of the third apparatus;

FIG. 14 illustrates a rear perspective view in partial cutaway of the third apparatus assembly;

FIG. 15 illustrates the perspective view of the forth apparatus of orthotic assembly providing mobility to the user with a knee injury;

FIG. 16 illustrates an enlarged perspective view in partial cutaway of the orthotic assembly of FIG. 15;

FIG. 17 illustrates a local perspective view of a first apparatus assembly for selectively separating the leg brace component from the foot bed.

FIG. 18 illustrates a local perspective view of a second apparatus assembly for selectively separating the leg brace component from the foot bed.

### DETAILED DESCRIPTION

An injury to the bottom surface of the foot can present additional problems for the user who desires to maintain mobility without the aid of crutches.

The use of crutches can allow the user some mobility after an injury to the foot has occurred. However, using crutches generally involve some training and are generally unwieldy to use. Furthermore as in re, stowing the crutches may present additional problems.

Amputees face similar problems to those described above. After a leg has been removed and the skin has healed over, the end of the leg remain sensitive to pressure due to the severed nerve endings. The sensitivity prevents a prosthesis from being used by the user until a significant period of time has passed.

The present invention eliminates the need for crutches for injuries to the foot and leg because the foot and leg brace system of the present invention employs calf support members in order to carry the weight of the user that would have normally been transferred to the foot. This allows the foot to be suspended without touching the ground or sole of any shoe. Furthermore, amputees do not have any pressure placed on the bottom of their leg and consequently do not suffer from this pressure.

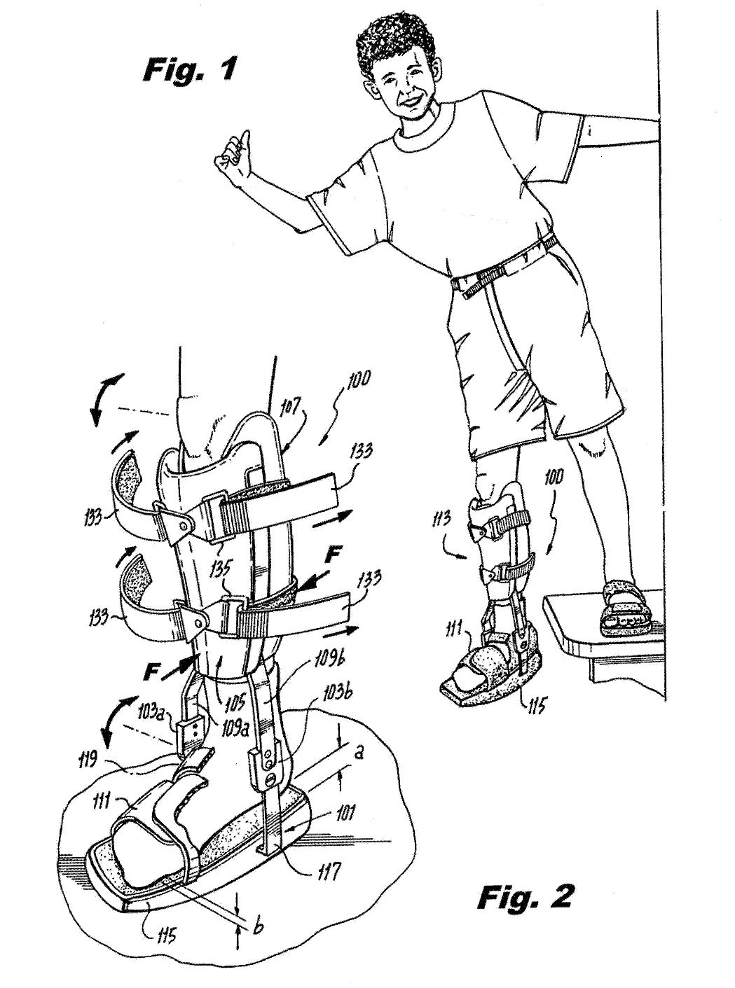

FIG. 1 illustrates the orthotic assembly 100 of the present invention supporting a leg and foot of a user. The leg brace system 100 may include a shoe member 111 to cooperate with the foot of the user and a calf member 113 to cooperate with the calf and lower leg of the user in order to support the weight of the user in order that the foot of the user may be maintained in a spaced relationship with the sole member 115 of the shoe member 111. The sole member 115 generally extends substantially perpendicular to the calf member 113 and generally maintains a spaced relationship with the foot of the user.

FIG. 2 illustrates a perspective and partially cutaway view of the leg brace system 100 of the present invention.

FIG. 2 illustrates that the shoe member 111 may include a substantially U shaped support member 101 which may include a first arm member 117 which may be upwardly extending and may be positioned in a spaced relationship with the side surface of the shoe member 111 and sole member 115 and which may include a near identical second arm member 119 which may be positioned on the opposing side surface of the shoe member 111 and sole member 115. The first arm member 117 and the second arm member 119 should be spaced with the shoe member 111 so that contact is avoided between the first arm member 117 and the second arm member 119 and the ankle and lower leg of the user.

The arm members 117, 119 may be detachably connected to the connection members 103a,b with for example screws or bolts or other appropriate fastening devices.

The connection members 103a,b may be detachably connected to support members 109a,b which extends substantially vertically to support and connect the shoe member 111 with the calf member 113 and which may be rigid. The connection members 103a,b may be rigidly connected to the support members 109a,b by screws or bolts or other appropriate fastening devices.

The calf member 113 may include a first calf support member 105 which may be a rigid member which may be formed from plastic, metal, wood or other material. The first calf support member 105 may be approximately U-shaped and may be concavely curved in order to cooperate with the front of the calf (the shin) of the user, and the first calf support member 105 may include a edge channels 131a,b which may be a near vertical channel formed on each side of the first calf support member 105 in order to cooperate with the upper portion of the support members 109a,b so that the inner surface of the first calf support member 105 cooperates with the inner surface of the second calf support member 107 in order to provide a near continuous inner surface in order to surround the calf of the user with support. The second calf support member 107 may be substantially a U-shaped member and may be concavely curved and may be formed from a rigid material such as plastic, wood, metal or other appropriate material. The second calf support member 107 may be affixed to the support members 109a,b so that the second calf support member 107 may be detachably connected to the first calf support member 105 by virtue of the support member 107 being positioned within the edge channels 131a,b. The edge channels 131a,b may extend from the bottom of the first calf support member 105 to the top of or near top of the first calf support member 105. The first calf support member 105 may be clamped/cinched/tied and secured to the second calf support member 107 by support straps 133 which may be affixed to the second calf support member 107 and a ring 135 or buckle may be affixed to the outer surface of the first calf support member 105 in order that one end of the each of the support straps 133 may be detachably connected. A portion of one end of the support straps 133 may include Velcro which may include hooks and loops in order to secure respective ends of the support straps 133. The user may connect the support straps 133 with sufficient force (F) in order that the first calf support member 105 and the second calf support member 107 may securely circumferentially compress around the calf of the user so that the weight of the user is offloaded onto support members 109a,b and transferred through the connector members 103a,b into the U-shaped member 101 within the foot bed. When connected, the first calf support member 105 and the second calf support member 107 includes an inner surface which may be frusto-conical which may be cone shaped but with the tip of the cone truncated. The inner surface of the connected first calf support member 105 and the second calf support member 107 may have inwardly sloping sides in order to provide a diminishing diameter as the bottom of the first calf support member 105 and the second calf support member 107 is approached. The inner surfaces of the first calf support member 104 and the second calf support member 107 may be formed to correspond to the shape of the calf.

By appropriate dimensions of the support members 109a,b and other elements, FIG. 2 illustrates an advantage of the invention in that the bottom surface of the heel of the user's foot is maintained at a distance ‘a’ from the top surface of the sole member 115 even while the user is applying the user's weight by walking and the bottom surface of the toes of the user's foot is maintained at a distance ‘b’ from the top surface of the sole member 115. These two distances namely distance ‘a’ and distance ‘b’ may be maintained even when the full weight of the user is placed upon the leg because the weight of the user is supported by the first calf support member 105 and the second calf support member 107 into the foot bed.

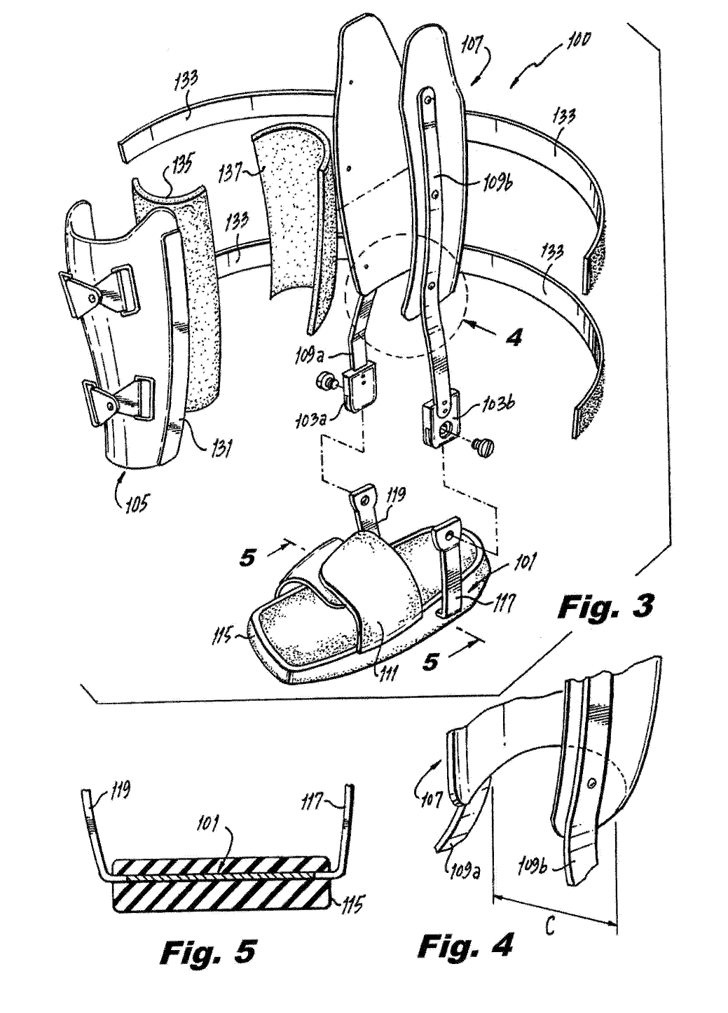

FIG. 3 is an exploded view and illustrates that the shoe member 111 may include a substantially U shaped support member 101 which may include a first arm member 117 which may be upwardly extending and may be positioned in a spaced relationship with the side surface of the shoe member 111 and sole member 115 and which may include a near identical second arm member 119 which may be positioned on the opposing side surface of the shoe member 111 and sole member 115. The first arm member 117 and the second arm member 119 may be spaced with the shoe member 111 so that contact is avoided between the first arm member 117 and the second arm member 119 and the ankle and lower leg of the user.

The arm members 117, 119 may be detachably connected to the connection members 103a,b with for example screws or bolts or other appropriate fastening devices.

The connection members 103a,b may be detachably connected to the support members 109a,b which extends substantially vertically to support and connect the shoe member 111 with the calf member 113 and which may be rigid. The connection members 103a,b may be connected to the support members 109a,b by screws or bolts or other appropriate fastening devices.

The calf member 113 may include a first calf support member 105 which may be a rigid member which may be formed from plastic, metal, wood or other material. The first calf support member 105 may be approximately U-shaped and may be concavely curved in order to cooperate with the front of the calf of the user, and the first calf support member 105 may include a edge channels 131a,b which may be a near vertical channel formed on each side of the first calf support member 105 in order to cooperate with the upper portion of the support members 109a,b so that the inner surface of the first calf support member 105 cooperates with the inner surface of the second calf support member 107 in order to provide a near continuous inner surface in order to surround the calf of the user with support. The second calf support member 107 may be substantially a U-shaped member and may be concavely curved and may be formed from rigid material such as plastic, wood, metal or other appropriate material. The second calf support member 107 may be affixed (or detachably connected) to the support members 109a,b so that the second calf support member 107 may be detachably connected to the first calf support member 105 by virtue of the support member 107 being positioned within the edge channels 131a,b. The edge channels 131a,b may extend from the bottom of the first calf support member 105 to the top of or near top of the first calf support member 105. The first calf support member 105 may be clamped/tied and secured to the second calf support member 107 by support straps 133 or clamps which may be affixed to the second calf support member 107 and a ring 135 or buckle may be affixed to the outer surface of the first calf support member 105 in order that one end of the each of the support straps 133 may be detachably connected. A portion of one end of the support straps 133 may include Velcro which may include hooks and loops in order to secure respective ends of the support straps 133. The user may connect the support straps 133 with sufficient force (F) in order that the first calf support member 105 and the second calf support member 107 may securely circumferentially compress around the calf of the user so that the weight of the user is offloaded onto support members 109a,b and transferred through the connector members 103a,b into the U-shaped member 101 within the foot bed. When connected, the first calf support member 105 and the second calf support member 107 includes a inner surface which may be frusto-conical which may be cone shaped but with the tip of the cone truncated. The inner surface of the connected first calf support member 105 and the second calf support member 107 may have inwardly sloping sides in order to provide a diminishing diameter as the bottom of the first calf support member 105 and the second calf support member 107 is approached. The inner surfaces of the first calf support member 104 and the second calf support member 107 may be formed to correspond to the shape of the calf.

FIG. 3 additionally illustrates a first removable cushion member 137 and a second removable cushion member 139 in order to cushion and conform the shape of the calf of the user to prevent chafing and assist in achieving a sufficient fit between the calf of the user and the first and second calf support member 105, 107. Different sizes of the first and second removable cushion members may be employed for different size legs and to provide flexibility for changes in size of the legs. The first cushion member 135 may be detachably connected to the interior surface of the first calf support member 105 by Velcro double-sided tape or other devices. In a similar fashion, the second cushion member 137 may be detachably connected to the interior surface of the second calf support member 107 by Velcro, double-sided tape or other devices.

FIG. 3 additionally illustrates a pair of foot straps 141 which extend across the foot and may be detachably connected by a fastening apparatus such as Velcro which may include hooks and loops.

FIG. 4 illustrates the bottom portion of the second calf support member 107 and illustrates that the second calf support member 107 extends inwardly or is concavely shaped as the bottom of the second calf support member 107 is approached. The second calf support member 107 includes a front vertical slot to cooperate with the first calf support member 105 and a bottom aperture with a dimension ‘c’ for the leg of the user to extend through. The width of dimension can be selectively determined during manufacture to provide calf support members 107 sized to fit calves of varies diameters. FIG. 4 additionally illustrates that the support members 109a,b conforms to the side of the second calf support member 107 and extends outwards to provide additional clearance for the leg of the user.

FIG. 5 illustrates that the base of the U shaped support member 101 is contiguous through the shoe member 111 and the first arm member 117 and the second arm member 119 extend upwards and in a spaced relationship with the shoe member 111.

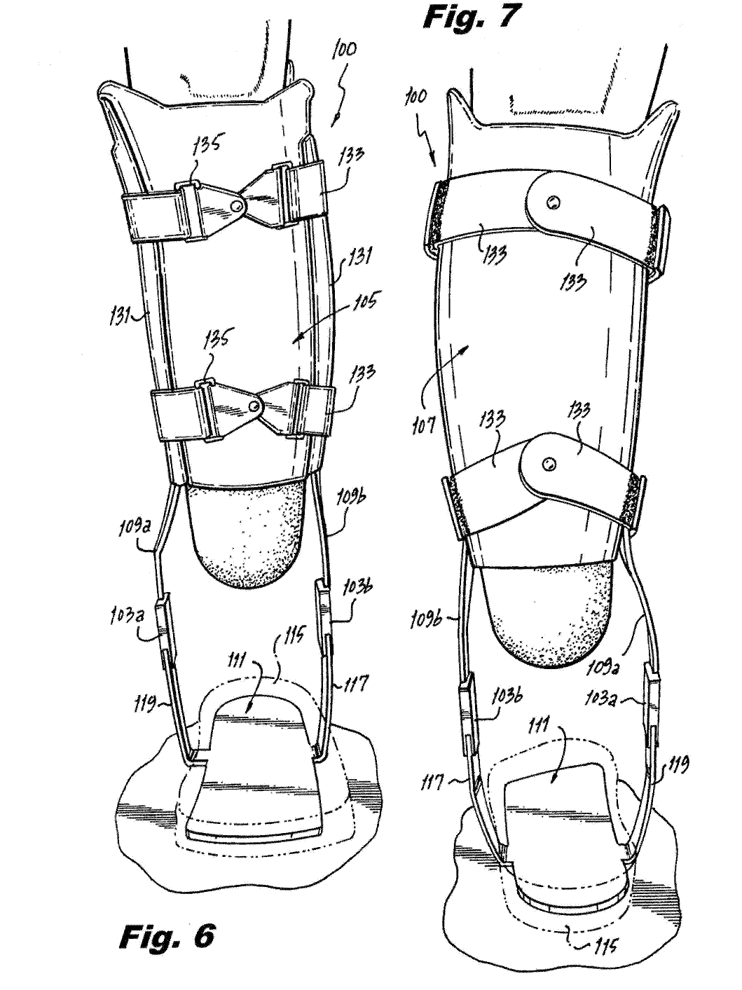

FIGS. 6 and 7 illustrates the installation of the orthotic assembly on an amputee. The end of the leg is free from touching any surface and the weight of the leg is therefore completely offloaded consequently the pain should be minimized or eliminated. The weight of the user may be transmitted to the first and second calf support members 107, 105 which may transfer the weight to the support members 109a,b which may transmit the weight to the connection members 103a,b and to the C shaped support member 101 through the first and second arm members 117, 119 to the shoe member 111 which transfers the weight to the surface of the ground.

FIG. 6 illustrates the ring 135/buckle affixed to the front surface of the first calf support member 105 and the ring 135 provides a detachable connection to the support straps 133.

FIG. 7 illustrates a rear elevation view of the orthotic assembly of FIG. 6 illustrating the fixturing means of support straps 133 are affixed to the front surface of the rear calf support member 107 and the securement of said straps 133 utilizing Velcro.

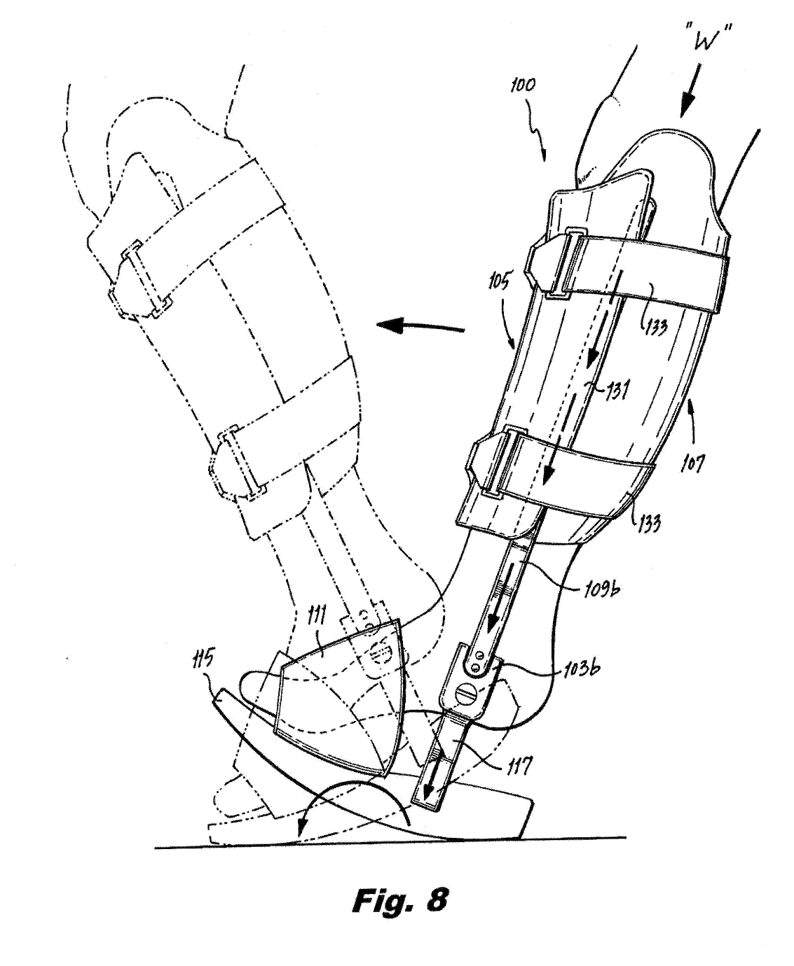

FIG. 8 illustrates a diagrammatic elevation view of the foot and ankle completely offloaded of weight as the user strides forward. During the operation the users weight “w” is transferred down the supports 109a,b and into shoe member 111. The bottom of the foot of the user does not touch the top surface of the shoe member 111 and maintains a spaced relationship with the top surface regardless of the shoe being in the first position or the second position. Because the shoe member 111 is provided with a convex underside surface, the shoe member 111 roles through the stride providing both safety and comfort. In certain instances, connectors 103a,b maybe permitted to allow rotation of shoe member 111. This maybe desirable as a patient convalesces and is permitted to place onto the joint incrementally.

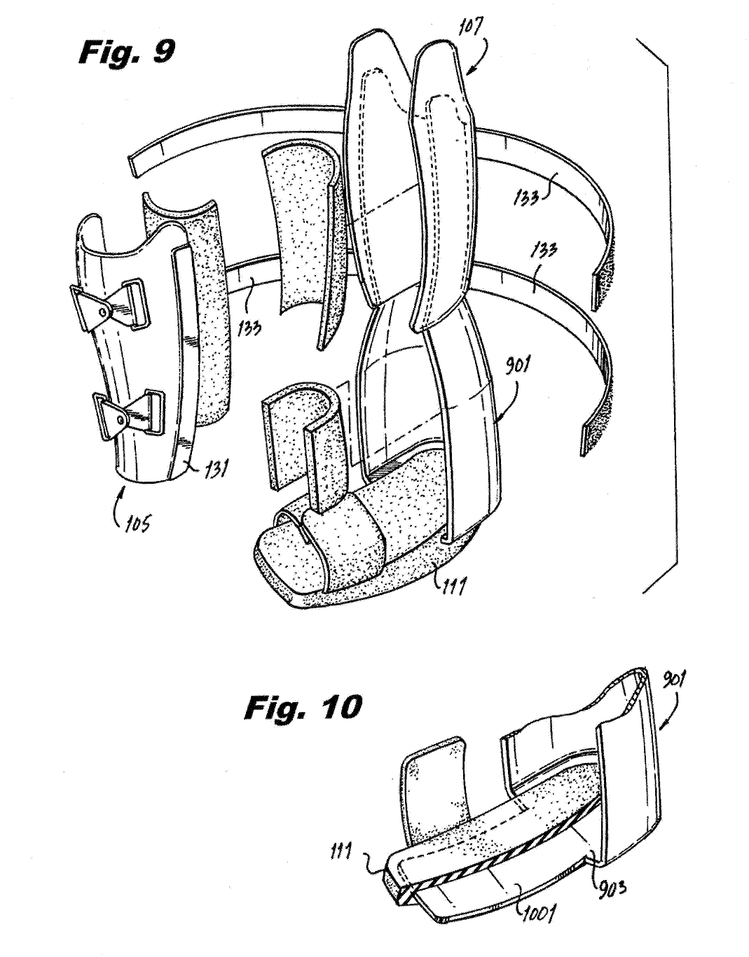

FIG. 9 illustrates a second apparatus wherein weight bearing member 111 may include an ankle support member 901 is substantially one piece and may have a second support member 107 molded onto the upper surface, or formed around it. This one piece member 901 eliminates the necessity of twin support members 109a,b and may further serve to shroud or shield an injured ankle during convalescence and maybe integrally formed with member 901. The ankle support member may be rigid and formed from plastic, wood, metal or other appropriate material and may mate with second calf support member 101. The ankle support member 901 may be concavely shaped and the increase in diameter towards the distal end of the ankle support member 901. The ankle support member 901 may include a base member 903 which extends through the shoe member 111. The sides of the ankle support member 901 may be positioned in a spaced relationship to the sides of the shoe member 111.

The ankle support member 901 may include a front vertical slot to cooperate with the slot of the second calf support member 107 for the leg of the user to extend through.

The calf member 113 may include a first calf support member 105 which may be a rigid member which may be formed from plastic, metal, wood or other material. The first calf support member 105 may be approximately U-shaped and may be concavely curved in order to cooperate with the front of the calf of the user, and the first calf support member 105 may include a edge channels 131a,b which may be a near vertical channel formed on each side of the first calf support member 105 in order to cooperate with the edge of the second calf support member so that the inner surface of the first calf support member 105 cooperates with the inner surface of the second calf support member 107 in order to provide a near continuous inner surface in order to surround the calf of the user with support. The second calf support member 107 may be substantially a U-shaped member and may be concavely curved and may be formed from rigid material such as plastic, wood, metal or other appropriate material. The second calf support member 107 may be detachably connected to the first calf support member 105 by virtue of the edge of the second calf support member 107 being positioned within the edge channels 131a,b. The edge channels 131a,b may extend from the bottom of the first calf support member 105 to the top of or near top of the first calf support member 105. The first calf support member 105 may be clamped/tied and secured to the second calf support member 107 by support straps 133 which may be affixed to the second calf support member 107 and a ring 135 or buckle may be affixed to the outer surface of the first calf support member 105 in order that one end of the each of the support straps 133 may be detachably connected. A portion of one end of the support straps 133 may include Velcro which may include hooks and loops in order to secure respective ends of the support straps 133. The user may connect the support straps 133 with sufficient force in order that the first calf support member 105 and the second calf support member 107 may securely hold the calf of the user so that the weight of the user is placed on the first calf support member 105 and the second calf support member 107. When connected, the first calf support member 105 and the second calf support member 107 includes a inner surface which may be frusto-conical which may be cone shaped but with the tip of the cone truncated. The inner surface of the connected first calf support member 105 and the second calf support member 107 may have inwardly sloping sides in order to provide a diminishing diameter in the distal direction of the first calf support member 105 and the second calf support member 107. The inner surfaces of the first calf support member 104 and the second calf support member 107 may be formed to correspond to the shape of the calf.

FIG. 10 illustrates the shoe member 111 and a portion of the ankle support member 901 and illustrates that the base member 903 of the ankle support member 901 includes a contiguous forward projecting member 1001 which may be rigid to provide weight bearing means for the shoe member 111 and to provide support for the ankle support member 901.

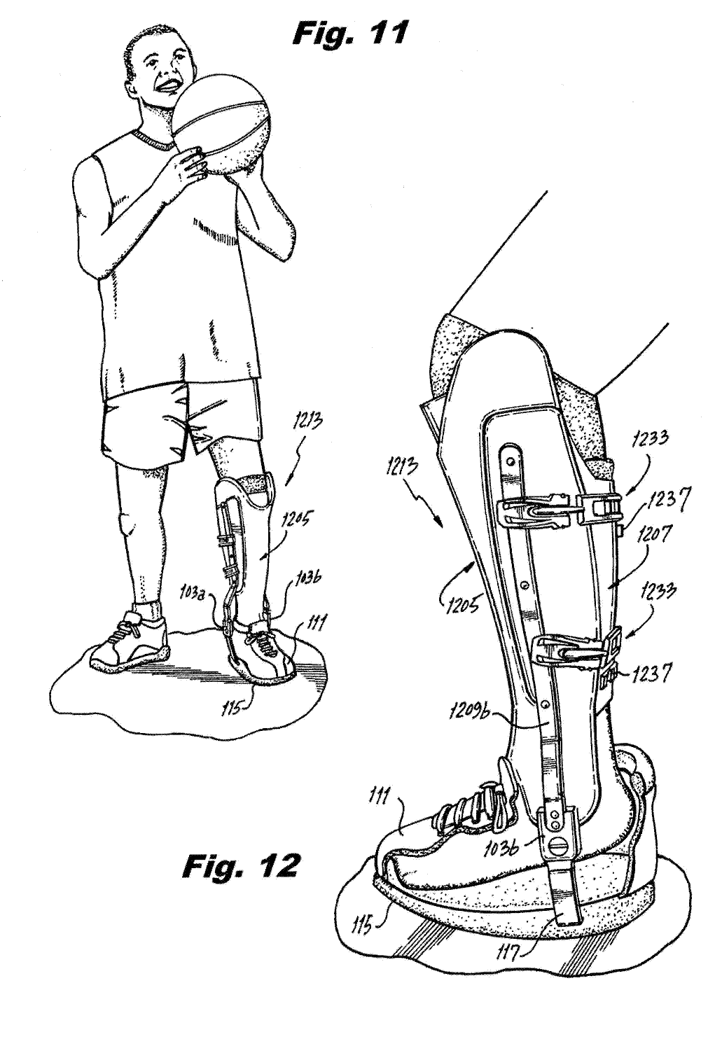

FIG. 11 illustrates that the shoe member 111 may include a substantially C shaped support member 101 which may include a first arm member 117 which may be upwardly extending and may be positioned in a spaced relationship with the side surface of the shoe member 111 and sole member 115 and which may include a near identical second arm member 119 which may be positioned on the opposing side surface of the shoe member 111 and sole member 115. The first arm member 117 and the second arm member 119 should be spaced with the shoe member 111 so that contact is avoided between the first arm member 117 and the second arm member 119 and the ankle and lower leg of the user.

The arm members 117, 119 may be detachably connected to the connection members 103a,b with for example screws or bolts or other appropriate fastening devices.

The connection members 103a,b may be detachably connected to support members 1209 which extends substantially vertically to support and connect the shoe member 111 with the calf member 2113 and which may be rigid. The connection members 103a,b may be connected to the support member 1209 by screws or bolts or other appropriate fastening devices.

The calf member 2113 may include a first calf support member 1205 which may be a rigid member which may be formed from plastic, metal, wood or other material. The first calf support member 1205 may be approximately U-shaped and may be concavely curved in order to cooperate with the front of the calf of the user, and the first calf support member 1205 may extend to the shoe member 111 and may be connected to the support member 1209. The inner surface of the first calf support member 1205 cooperates with the outer surface of the second calf support member 1207 in order to provide a near continuous inner surface in order to surround the calf of the user with support. The second calf support member 1207 may be substantially a U-shaped member and may be concavely curved and may be formed from rigid material such as plastic, wood, metal or other appropriate material. The second calf support member 1207 may not extend to the shoe member 111. The second calf support member 1207 may be detachably connected to the first calf support member 1205. The first calf support member 1205 may be clamped/tied and secured to the second calf support member 107 by a second portion 1235b of a latch 1233 which may be affixed to the second calf support member 107, and a first portion of a latch 1235a or buckle may be affixed to the outer surface of the first calf support member 1205 in order that the first calf support member 1205 may be detachably connected to the second calf support member 1207. The latch 1233 may include multiple latching positions so that the positioning of the first calf support member 1205 may be adjusted with respect to the second calf support member 1207. The user may connect the latch 1233 sufficiently so that the first calf support member 1205 and the second calf support member 1207 may securely circumferentially compress around the calf of the user so that the weight of the user is offloaded onto support members 109a,b and transferred through the connector members 103a,b into the U-shaped member 101 within the foot bed. The latches 1233 may be detachably connected to the support member 1209 which may include multiple apertures 1237 to allow the adjustment of the latches 1233 to different positions. When connected, the first calf support member 1205 and the second calf support member 1207 includes an inner surface which may be frusto-conical which may be cone shaped but with the tip of the cone truncated. The inner surface of the connected first calf support member 1205 and the second calf support member 1207 may have inwardly sloping sides in order to provide a diminishing diameter in the distal direction. The inner surfaces of the first calf support member 1205 and the second calf support member 1207 may be formed to correspond to the shape of the calf.

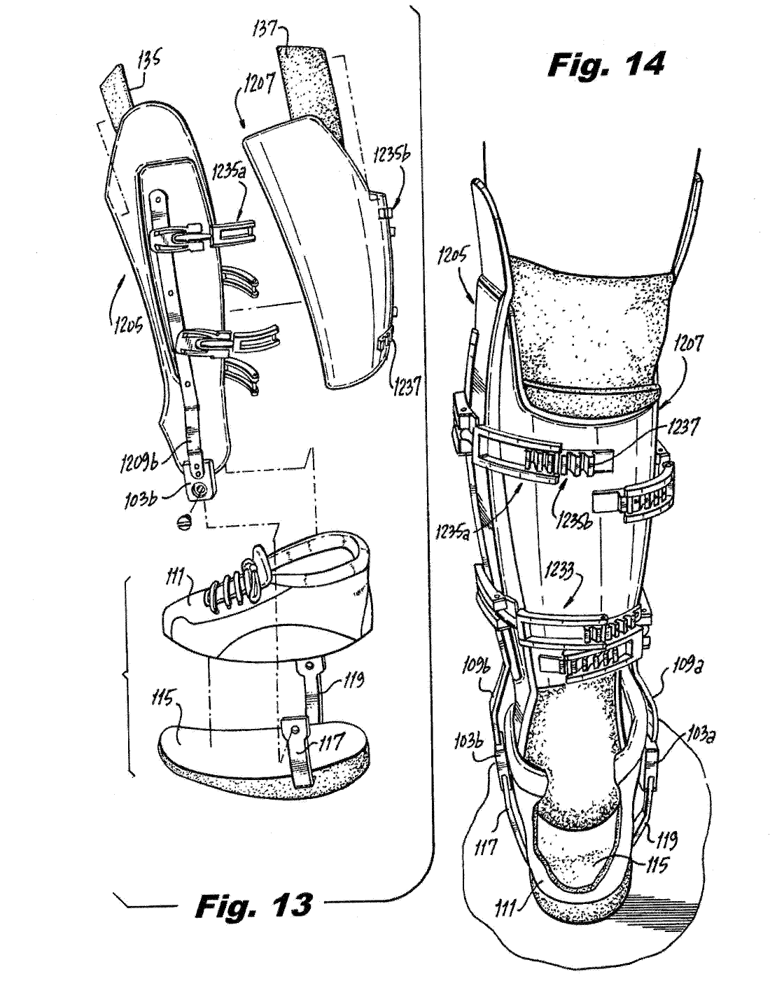

FIG. 13 is an exploded view of the components of the foot and leg brace system and illustrates that the shoe member 111 may include a substantially C shaped support member 101 which may include a first arm member 117 which may be upwardly extending and may be positioned in a spaced relationship with the side surface of the shoe member 111 and sole member 115 and which may include a near identical second arm member 119 which may be positioned on the opposing side surface of the shoe member 111 and sole member 115. The first arm member 117 and the second arm member 119 should be spaced with the shoe member 111 so that contact is avoided between the first arm member 117 and the second arm member 119 and the ankle and lower leg of the user.

The arm members 117, 119 may be detachably connected to the connection members 103a,b with for example screws or bolts or other appropriate fastening devices.

The connection members 103a,b may be detachably connected to the support member 1209a,b which extends substantially vertically to support and connect the shoe member 111 with the calf member 1213 and which may be rigid. The connection members 103a,b may be connected to the support member 1209a,b by screws or bolts or other appropriate fastening devices.

The calf member 2113 may include a first calf support member 1205 which may be a rigid member which may be formed from plastic, metal, wood or other material. The first calf support member 1205 may be approximately U-shaped and may be concavely curved in order to cooperate with the front of the calf of the user, and the first calf support member 1205 may extend to the shoe member 111 and may be connected to the support member 1209. The inner surface of the first calf support member 1205 cooperates with the inner surface of the second calf support member 1207 in order to provide a near continuous inner surface in order to surround the calf of the user with support. The second calf support member 1207 may be substantially a U-shaped member and may be concavely curved and may be formed from rigid material such as plastic, wood, metal or other appropriate material. The second calf support member 1207 may not extend to the shoe member 111. The second calf support member 1207 may be detachably connected to the first calf support member 1205. The first calf support member 1205 may be clamped/tied and secured to the second calf support member 107 by a second portion of a latch 1235a,b which may be affixed to the second calf support member 107 and a first portion of a latch 1235a or buckle may be affixed to the outer surface of the first calf support member 1205 in order that the first calf support member 1205 may be detachably connected to the second calf support member 1207. The latch 1233 may include multiple positions so that the positioning of the first calf support member 1205 may be adjusted with respect to the second calf support member 1207. The user may connect the latch 1233 sufficiently so that the first calf support member 1205 and the second calf support member 1207 may securely hold the calf of the user so that the weight of the user is placed on the first calf support member 1205 and the second calf support member 1207. The latches 1235a may be pivotally connected to the support member 1209 which may include multiple projections or teeth 1237 to allow the adjustment of the latches 1233 two different positions. When connected, the first calf support member 1205 and the second calf support member 1207 includes an inner surface which may be frusto-conical which may be cone shaped but with the tip of the cone truncated. The inner surface of the connected first calf support member 1205 and the second calf support member 1207 may have inwardly sloping sides in order to provide a diminishing diameter in the distal direction. The inner surfaces of the first calf support member 1205 and the second calf support member 1207 may be formed to correspond to the shape of the calf.

FIG. 14 illustrates the compression of a patient lower leg within members 1207 and 1208 via latch assemblies 1233. The heel of the patient's foot is shown in spaced relation to sole bed 115.

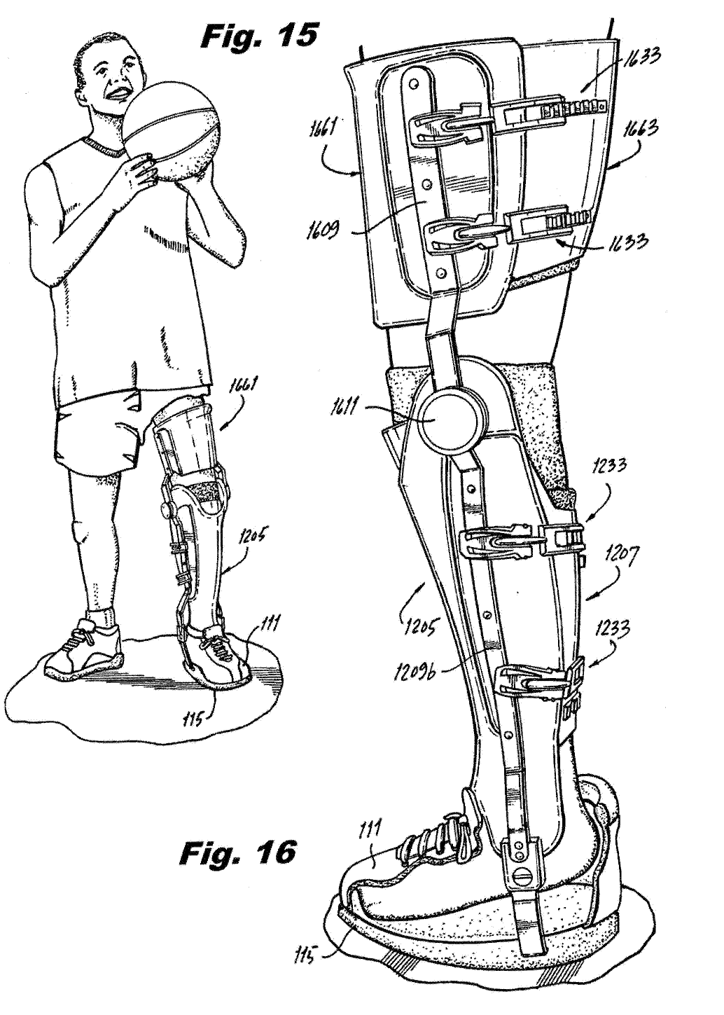

FIG. 15 illustrates a forth apparatus of the present invention including a thigh support member to provide for offloading the patients knee.

FIG. 16 illustrates a side view of the foot and leg brace system which may include a first thigh support member 1661 and a second thigh support member 1663 which may be connected together with a latch 1633. A thigh support member 1609 may be connected to the first thigh support member 1661 and may be connected to a pivot member 1611 which allows the thigh support member 1609 to pivot with respect to the support member 1209. The thigh support member 1609 may cooperate with and near identical thigh support member 1609 which may be positioned on the opposing side of the first thigh support member 1661. The first thigh support member 1661 and the second thigh support member 1663 may cooperate with the first calf support member 1205 and the second calf support member 1207 in order to stabilize the leg and support the leg of the user.

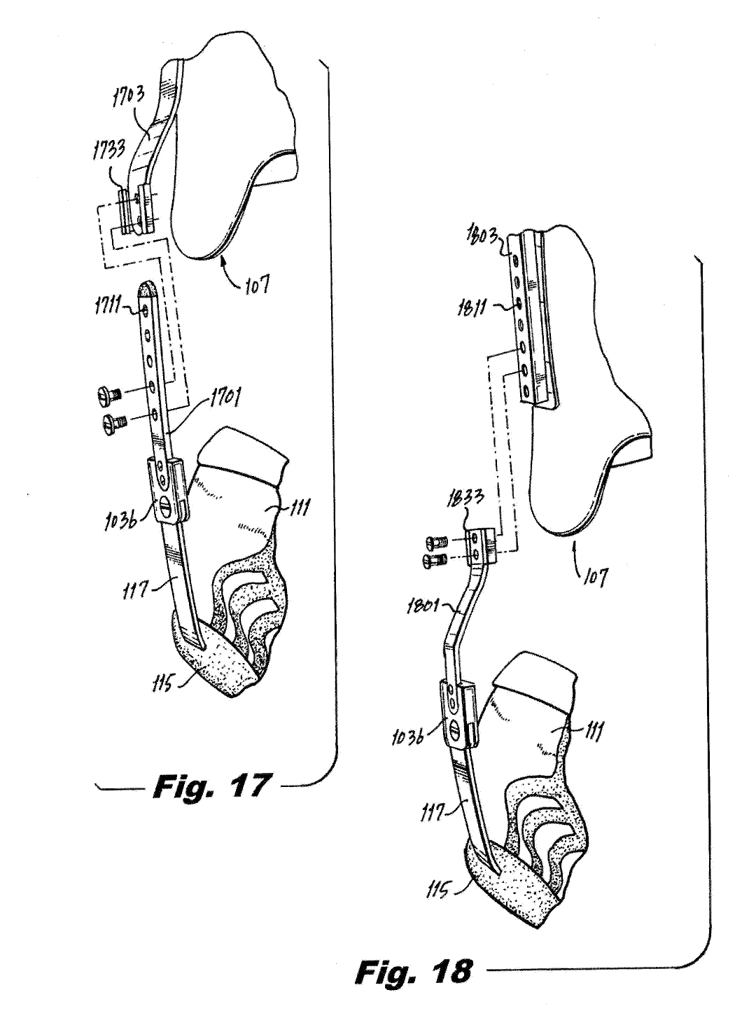

FIG. 17 illustrates a portion of the second calf support member 107 and a portion of the shoe member 111 which may include the first arm member 117 and the connection members 103a,b.

FIG. 17 illustrates that the support member 1709 may be separated into a first portion 1701 of the support member 1709 and a second portion 1703 of the support member 1709. The first portion 1701 may include multiple apertures 1711 to adjust the relative distance between the first calf support member 105 and the shoe member 111 and the second portion 1703 may include a connection member 1733 to detachably connect to the first portion 1701. The connection member 1733 may include screws or bolts or other connection devices to connect to the first portion 1701.

FIG. 18 illustrates that the support member 1809 may be separated into a first portion 1801 of the support member 1809 and a second portion 1803 of the support member 1809. The second portion 1803 may include multiple apertures 1811 to adjust the relative distance between the first calf support member 105 and the shoe member 111 and the first portion 1801 may include a connection member 1833 to detachably connect to the second 1803. The connection member 1833 may include screws or bolts or other connection devices to connect to the second portion 1803.

While the invention is susceptible to various modifications and alternative forms, specific apparatuss thereof have been shown by way of example in the drawings and are herein described in detail. It should be understood, however, that the description herein of specific apparatuss is not intended to limit the invention to the particular forms disclosed.

1. An apparatus for supporting a calf of a user, comprising:
a first calf support member to support the calf of the user;
a second calf support member to support the calf of the user;
a shoe member to support the first and second calf support member;
wherein the first calf support member and the second calf support member cooperate to form a substantially continuous inner surface to support the calf of the user.

2. An apparatus for supporting a calf of a user as in claim 1, wherein the first calf support member includes a slot to accept the leg of the user.

3. An apparatus for supporting a calf of a user as in claim 1, wherein the second calf support member includes a slot to accept the leg of the user.

4. An apparatus for supporting a calf of a user as in claim 1, wherein the first calf support member and the second calf support member are detachably connected with a strap.

5. An apparatus for supporting a calf of a user as in claim 1, wherein the first calf support member and the second calf support member are detachably connected with a latch.

6. An apparatus for supporting a calf of a user as in claim 4, wherein the strap includes a Velcro connection device.

7. An apparatus for supporting a calf of a user as in claim 1, wherein the first calf support member includes a removable cushion member to cushion the calf of the user.

8. An apparatus for supporting a calf of a user as in claim 1, wherein the second calf support member includes a removable cushion member to cushion the calf of the user.

9. An apparatus for supporting a calf of a user as in claim 1, wherein the shoe member and the first calf support member are connected with a support member.

10. An apparatus for supporting a calf of a user as in claim 1, wherein the shoe member and the second calf support member are connected with a support member.

11. An apparatus for supporting a calf of a user as in claim 1, wherein the second calf support member does not extend to the shoe member.

12. An apparatus for supporting a calf of a user as in claim 1, wherein the second calf support member extends to the shoe member.

13. An apparatus for supporting a calf of a user as in claim 1, wherein the apparatus includes a first thigh support member to support a thigh of the user.

14. An apparatus for supporting a calf of a user as in claim 13, wherein the apparatus includes a second thigh support member to support the thigh of the user and to cooperate with the first thigh support member.
